(19) United States
(12) Patent Application Publication
(10) Pub. No.: US 2005/0244387 A1
(43) Pub. Date: Nov. 3, 2005

(54) Entomopathogenic nematodes and methods of their use

(76) Inventor: Parwinder Grewal, Wooster, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/116,880

(22) Filed: Apr. 28, 2005

Related U.S. Application Data
(60) Provisional application No. 60/566,291, filed on Apr. 28, 2004.

(51) Int. Cl. A01K 67/00; A01K 67/033; A01N 65/00

(52) U.S. Cl. 424/93.7; 800/13

(57) ABSTRACT

An isolated nematode, wherein the nematode is Heterorhabditis bacteriophora strain GPS11, which was deposited at the ATCC, 10801 University Blvd., Manassas, Va. 20110-2209, on Apr. 22, 2004, under accession number ______. Methods of use of this nematode are also described.

This application claims priority to U.S. Provisional Patent Application No. 60/566,291, filed Apr. 28, 2004. The entire disclosure of this application is incorporated herein by reference.

Research leading to the present invention was funded, at least in part, by USDA Grant No. 00-35302-9336 and USDA PMAP Grant No. 00-34381-9556. The government has certain rights in this invention.

### DESCRIPTION OF THE INVENTION

1. Field of the Invention

The invention generally relates to a novel strain of entomopathogenic nematode belonging to Heterorhabditis bacteriophora, named GPS11. The GPS11 strain is equal or better than existing nematode strains in its virulence toward many insect species, and shows improved stress resistance over those other nematode strains. The invention is also directed to methods of use of GPS11 in controlling pest insects.

2. Background of the Invention

Entomopathogenic nematodes in the Steinernematidae and Heterorhabditidae (Order: Rhabditida) families are lethal parasites associated with symbiotic bacteria in the family Enterobacteriacae. Steinernematids are associated with Xenorhabdus spp. and heterorhabditids with Photorhabdus spp. bacteria (Boemare, 2002; Forst and Clark, 2002). The only free-living stage of the nematode is the infective juvenile (IJ), also referred to as ‘dauer’ (enduring) juvenile, which is capable of actively seeking out hosts (Kaya and Gaugler, 1993). Following entry through natural body openings, and in some cases through the cuticle, the IJs release the symbiotic bacteria into the insect hemocoel (Poinar, 1990). The bacteria then multiply rapidly, killing the host within 1-4 days. Nematodes complete 1-3 generations in the host cadaver and new infective juveniles are produced as food resources are depleted. The IJs leave the cadaver to seek new hosts. Thus, entomopathogenic nematodes have potential to recycle and provide long-term pest control.

While entomopathogenic nematodes are available, there is still a need for more stress-resistant strains. The present invention advances the field by providing a new strain of nematode that exhibits excellent virulence combined with improved stress-resistance.

### SUMMARY OF THE INVENTION

Features and Advantages

In accordance with the invention, a novel nematode strain is provided. The nematode is an entomopathogenic nematode belonging to Heterorhabditis bacteriophora that has superior combination of characteristics important to biological pest control. The new strain is designated GPS11, and is characterized by unique isozyme patterns of several enzymes, by superior storage stability of infective juveniles (over 50% survival for over nine weeks at 25° C. in water with 10% of the nematodes surviving over twelve weeks), high heat tolerance (over 90% survival at 40° C. for two hours), high desiccation tolerance (over 80% survival in 25% glycerol solution), and high virulence potential against white grubs (LD₅₀ of less than 550 infective juveniles per last instar Japanese beetle in pot assays). The new strain also has high virulence toward the black vine weevil (Otiorhynchus sulcatus), grape root borer (Vitacea polistiformis), and fungus gnats (Bradysia spp.), and other major pests.

The infective juvenile longevity of the new strain in water at 25° C. is 34 times longer than the commercially available HP88 strain of H. bacteriophora. The GPS11 strain is also superior to HP88 strain in heat tolerance (five times higher survival than HP88 strain at 40° C. for two hours), UV tolerance (twice as good as HP88), and hypoxia (twice as good as HP88). GPS11 is also more virulent than the HP88 strain against the economically important white grub species of Japanese beetle (Popillia japonica) and Oriental beetle (Anomala (Exomala) orientalis). In fact, GPS11 is more virulent against the Japanese beetle than all the available strains and species of entomopathogenic nematodes except the X1 strain of H. zealandica, which has poor storage stability. The only commercial product based on H. zealandica has no shelf-life at 5° C. and has only one week of shelf-life at room temperature, severely limiting its commercial potential. GPS11 is at least four times superior to H. zealandica in storage stability, and the strain is equally effective in controlling white grubs in the field. The combination of characteristics makes GPS11 extremely desirable.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

### SUMMARY OF THE INVENTION

The present invention provides an isolated nematode, wherein the nematode is Heterorhabditis bacteriophora strain GPS11, which was deposited at the ATCC, 10801 University Blvd., Manassas, Va. 20110-2209, on Apr. 22, 2004, under accession number ______, or progeny of said nematode.

The present invention also provides insecticidal compositions comprising at least one GPS11 nematode and at least one agriculturally acceptable carrier component. In some embodiments, the carrier component comprises water. In some embodiments, the carrier component further comprises at least one polymer, which can be chosen from agaroses, carbopols, carrageenans, dextrins, gums, starches, alginates, acrylamides, and glutens. In some embodiments, the carrier component further comprises at least one humectant. The carrier component can comprise at least one humectant and at least one polymer.

The present invention also provides methods of controlling a population of insect pests, comprising applying an effective amount of the GPS11 nematode, to an affected area. The insect pests to be controlled include armyworms, cutworms, sod webworms, fleas, mole crickets, crane flies, weevils, white grubs, fungus gnats, and onion maggots.

It is to be understood that both the foregoing general description and the following detailed description are exemplary and explanatory only and are not restrictive of the invention, as claimed. The accompanying drawings, which are incorporated in and constitute a part of this specification, serve to explain the principles of the invention.

### BRIEF DESCRIPTION OF THE DRAWINGS

FIG. 1 is a box plot showing overall field efficacy of the entomopathogenic nematodes and chemical insecticide, trichlorfon, applied curatively at standard rates against white grubs, Popillia japonica and Cyclocephala borealis based on pooled data from all the trials during 2000. A. Combined analysis for all trials; B. Combined analysis for all trials in which the total amount of post-application irrigation plus rainfall was optimum for nematode efficacy.

FIG. 2 shows the mean (±S.E.) percent mortality of 3^{rd} instar Popillia japonica (▪) and Cyclocephala borealis (□) produced by different species and strains of Heterorhabditis isolated from either within or outside the current geographic range of the two scarab species. Each grub was exposed to 200 infective juvenile nematodes in 28.9 ml plastic portion cups containing 20 g of heat-sterilized sand. Grub mortality was recorded after 2 weeks at 22° C. Same letter on the bar indicates no significant difference at P>0.05 for a nematode species or strain. Hb=H. bacteriophora, Hi=H. indica, Hma=H. marelatus, Hme=H. megidis, and Hz=H. zealandica.

FIG. 3 shows percent mortality of 3^{rd} instar Popillia japonica, Anomala orientalis, Rhizotrogus majalis, and Cyclocephala borealis at different concentrations of (A) Heterorhabditis bacteriophora strain GPS11, (B) H. bacteriophora strain HP88, (C) H. megidis strain UK, and (D) H. zealandica strain X1 after 2 weeks at 22° C. The grubs were individually exposed to nematodes in 148 ml plastic cups containing 80 g of heat sterilized sand.

FIG. 4 shows mean (±S.E.) number of nematodes penetrated (□) and encapsulated (▪) per 3^{rd} instar Popillia japonica or Cyclocephala borealis grub 24 h after exposure. Same letter(s) on the bar indicates no significant difference at P>0.05 for penetration or encapsulation.

FIG. 5 shows mean % survival (±S.E.) of dauer juveniles of H. bacteriophora populations after incubation at 40° C. for 2 h. Bars with the same letter(s) do not differ significantly at P=0.05.

FIG. 6 shows mean % survival (±S.E.) of dauer juveniles of H. bacteriophora populations under hypoxia for 96 h. Bars with the same letter(s) do not differ significantly at P=0.05.

FIG. 7 shows mean % survival (±S.E.) of dauer juveniles of H. bacteriophora populations after desiccation in 25% glycerol for 72 h. Bars with the same letter(s) do not differ significantly at P=0.05.

FIG. 8 shows mean % original virulence remaining (±S.E.) in dauer juveniles of H. bacteriophora populations following exposure to medium wave UV for 5 min. Bars with the same letter(s) do not differ significantly at P=0.05.

FIG. 9 shows regression analysis showing relationship of dauer juvenile longevity (LT₉₀) with heat tolerance, hypoxia tolerance, UV tolerance, and desiccation tolerance of dauer juveniles of H. bacteriophora populations.

FIG. 10 shows the first three principal components of the correlation matrix of longevity and tolerance to heat, UV, hypoxia, and desiccation.

FIG. 11 shows the percentage of grape root borer larvae infected by Heterorhabditis and Steinernema nematode species/strains. Percents followed by the same letter are not significantly different as determined by Pearson Chi-square (P=0.05). Hb=Heterorhabditis bacteriophora.

### DESCRIPTION OF THE EMBODIMENTS

Reference will now be made in detail to specific embodiments of the present invention, examples of which are illustrated in the accompanying drawings.

The present invention is generally directed to a new strain of Heterorhabditis bacteriophora, called GPS11, which exhibits many desirable characteristics. The new strain obtained from an infected white grub found in a golf course in Northeast Ohio. Given the source of the strain, finding the present strain again in nature may be difficult or impossible. For this reason, a deposit of the strain has been made with the ATCC, as detailed below.

The nematodes of the invention can be used in the control of a variety of insects, including but not limited to: armyworms (Noctuidae: Lepidoptera), including but not limited to, armyworm, fall armyworm, yellow-striped armyworm, and lawn armyworm; cutworms (Noctuidae: Lepidoptera), including but not limited to, black cutworms, bronze cutworms, variegated cutworms, and glassy cutworms; sod webworms (Pyralidae: Lepidoptera), including but not limited to, bluegrass webworm, larger sod webworm, Western lawn moth, striped sod webworm, elegant sod webworm, vagabond sod webworm, cranberry girdler, tropical sod webworm, and burrowing sod webworm; fleas (Policidae: Siphonaptera), including but not limited to, dog flea and cat flea; mole crickets (Gryllotaplidae: Orthoptera), including but not limited to, tawny mole cricket, Southern mole cricket, short winged mole cricket, and native mole cricket; crane flies or leather jackets (Tipulidae: Diptera), including but not limited to, European crane flies; weevils (Curculionidae: Coleoptera), including but not limited to, annual bluegrass weevil, black vine weevil, bluegrass billbug, hunting billbug, Phoenician billbug, and Denver billbug; white grubs (Scarabaeidae: Coleoptera), including but not limited to, Asiatic garden beetle, black turfgrass ataenius, green June beetle, Japanese beetle, May or June beetles, Oriental beetle, European chafer, Northern masked chafer, Southern masked chafer, and Southwestern masked chafer; and onion maggot.

The insects may be susceptible to infection and/or killing by the nematodes of this invention at several stages of their lives. For example, armyworms, cutworms, sod webworms, and fleas may be susceptible to infection at, for example, the larval and pupal stages; mole crickets may be susceptible to infection at the nymph and adult stages; crane flies and weevils may be susceptible at the larval and adult stages; and white grubs may be susceptible at the larval stage. Of course, the nematodes of the invention may infect insects at other stages, but these are the stages at which the insects are more susceptible.

Similarly, the nematodes of the invention are primarily infective during the only free-living stage of the nematode, i.e., the infective juvenile (IJ) stage, also referred to as ‘dauer’ (enduring) juvenile. The Heterorhabditis bacteriophora IJ is capable of actively seeking out hosts (Kaya and Gaugler, 1993). Following entry through natural body openings, and in some cases through the cuticle, the IJs release the symbiotic bacteria into the insect hemocoel (Poinar, 1990). The bacteria then multiply rapidly, killing the host within 1-4 days. Nematodes complete 1-3 generations in the host cadaver and new infective juveniles are produced as food resources are depleted. The IJs leave the cadaver to seek new hosts. Thus, entomopathogenic nematodes have potential to recycle and provide long-term pest control.

Because of their ability to reproduce and provide long-term protection, repeated administration of the nematodes of the invention may not be necessary. However, if desired, repeated administrations can be performed. The target area to be treated can be treated as often as multiple times in one day, to as infrequently as a single administration on a target area.

A target area can be an area infested with insect pests or an area prone to such insect infestation. Thus, the nematodes can be applied to areas in which insects are present or areas in which the insects are not yet present but may be present in the future. Control of the insect population may be designed to maintain the number of pests, i.e., prevent an increase, or to reduce the number of existing pests; or to prophylactically attenuate or prevent an infestation.

Administration of the compositions of the invention, which include the inventive nematodes, can be applied using any method. Liquid or gel compositions can be applied with spraying, using any known technique. Those of skill in the art will immediately recognize available techniques. For example, for compositions in the form of aqueous sprays, application can be carried out with typical agricultural and/or horticultural and/or arboricultural spraying equipment including pressurized fan sprayers, venturi sprayers, and boom sprayers. For compositions to be applied in a solid form, application may be carried out with typical agricultural and/or horticultural and/or arboricultural scattering equipment such as those used for spreading fertilizers on lawn.

It should be noted that this disclosure may refer to “agricultural” and/or “horticultural” and/or “arboricultural.” The three terms may be used interchangably, and no distinction is intended. Reference to any or all of the three terms is intended to encompass the growing of all types of plants, in any area, for any purpose, of any shape, size, or type.

In use, an insecticidally effective amount of the nematode of this invention is applied to the locus of, or in the vicinity of, the insects to be controlled. An “insecticidally effective amount,” as used herein, is the number of nematodes that will result in a desired infection or mortality rate of a group of insects. The actual effective amount can be readily determined by the practitioner skilled in the art, and can vary with the species of pest, stage of larval development, the type of vehicle or carrier, the period of treatment, environmental conditions (including moisture), and other related factors. Thus, an insecticidally effective amount can be empirically derived based on experience by the user.

In one embodiment, the nematodes are applied at a concentration greater than or equal to about 1.0×10⁴, 2.5×10⁴, 5.0×10⁴, 7.5×10⁴, 1.0×10⁵, 2.5×10⁵, 5.0×10⁵, 7.5×10⁵, or higher number of infective juveniles per m² of target area. Alternatively, the concentration of nematodes to be applied may also be determined relative to the density of the target insects, if known. The nematodes can be applied at a concentration of from about 10 nematodes per insect to about 10,000 nematodes per insect, or any number in between. Regardless of the method by which application concentration is decided, it may be desirable to follow the application with a heavy watering in order to soak the composition into the root zone where larval scarabs feed. In addition, it may be desirable to apply the nematode composition at dusk, to avoid unnecessary exposure to sunlight.

The term “controlling” as used herein in relation to a population of insect pests, is intended to refer to both maintaining (i.e. preventing increases) and reducing said population.

The nematodes of the invention can be used in compositions that include one or more suitable agricultural and/or horticultural carriers. Some compositions include water in a carrier, with a population of the nematodes suspended therein. In other embodiments, the carrier may include a solid phase material or encapsulating agent, upon or within which the nematodes can be immobilized. Suitable carriers of this type include but are not limited to hydrogel agents such as alginate gels, wheat-gluten matrices, starch matrices, wheat-bran bait pellets, clay particles, polyacrylamide gels, or synthetic polymers as are known in the art. Preferred alternative carriers and methods for immobilizing nematodes are described, for example, in Nelsen (U.S. Pat. Nos. 4,753,799; 4,701,326 and 4,615,883 disclosing alginate gels), Connick and Nickle (U.S. patent application Ser. No. 07/560,792, filed Jul. 30, 1990, disclosing wheat gluten), Shasha et al. (U.S. Pat. No. 4,859,377 disclosing starch matrices), and Capinera and Hibbard (J. Agric. Entomol., 4:337-344, (1987) disclosing wheat-bran bait pellets), the contents of each of which are incorporated herein by reference.

Formulations of alginate gels containing the nematodes provide the added benefit of enhanced viability after storage, while allowing subsequent conversion to an aqueous liquid by dissolution of the alginate with sodium citrate. When the carrier component includes elements in addition to water, sufficient moisture should be provided to ensure viability and infectivity of the nematodes. The entire composition can have a water activity greater than about 0.90, 0.91, 0.92, 0.93, 0.94, 0.95, 0.96, 0.97, 0.98, 0.99, or even greater.

In this regard, some embodiments include one or more components that are designed to control the water concentration of the composition. For example, in one embodiment, the substance is a water-retentive polymer. The water-retentive polymer can be dissolved in water or a water-based solution, such as a buffered solution, and then combined with the nematodes. In some embodiments, the water-retentive polymer is a gel-forming polymer. Examples of such gel-forming polymers include, but are not limited to, agarose, carbopols, carrageenan, dextrin, guar gum, and gellan gum. Gel-forming polymers can be liquid at room temperature and can be induced to gel by adding a gel inducing agent, such as for example an ion, to the formulation. A gel inducing agent can be added at the time the nematodes are combined with the carrier, or, alternatively later, such as for example, just prior to application of the formulation.

In one embodiment, the component for controlling water content is a humectant. Humectants include but are not limited to, sugars, carbohydrates, alcohols, polyhydric alcohols such as glycerol and sorbitol, and glycols and ether glycols such as mono- or diethers of polyalkylene glycol, mono- or diester polyalkylene glycols, polyethylene glycols (typically up to a molecular weight of about 600), glycolates, glycerol, sorbitan esters, esters of citric and tartaric acid, imidazoline derived amphoteric surfactants, lactams, amides, polyamides, quaternary ammonium compounds, esters such as phthalates, adipates, stearates, palmitates, sebacates, or myristates, glycerol esters, including mono/di/tri-glycerides, and combinations of any of the foregoing.

It should be noted that some humectants are polymers and that some polymers are humectants. These two groups are not intended to be mutually exclusive. In some embodiments, the formulation comprises both a water-retentive polymer and a humectant.

Because nematodes can be sensitive to UV light, it may be desirable to add UV protectants to the formulation. UV protectants include but are not limited to, acridine yellow, alkali blue, brilliant yellow, Congo red, lissamine green, mercurochrome, methylene blue, benzilidine sulfonic acid, Ulvinul DS49, Erio Acid Red, Raymix, and Tinopal.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The invention may be understood more clearly with the aid of the non-limiting examples that follow, and which constitute an advantageous embodiment of the compositions in accordance with the invention.

### EXAMPLES

### Example 1

### Obtaining GPS11 Strain

Heterorhabditis bacteriophora strain GPS11 was obtained from an infected white grub (C. borealis) from a soil sample obtained from a golf course in Northeast Ohio. It is noted that while this strain was isolated from nature, it may be difficult or impossible to obtain it again from nature. Thus, in an effort to guarantee its accessibility to the public, a deposit has been made in accordance with the Budapest Treaty.

The strain was deposited with the American Type Culture Collection, whose address is 10801 University Blvd., Manassas, Va. 20110-2209, on Apr. 22, 2004. The deposit been assigned accession number ______. The deposit was made in accordance with the Budapest Treaty.

### Example 2

### Biological Control of White Grubs with Entomopathogenic Nematodes

Nematodes and Grubs

Nematode species/strains and chemical insecticides used in this study are listed in Table 1.

TABLE 1

| Nematode/ | Abbreviation/ | |
|---|---|---|
| Insecticide | Formulation | Source |
| Nematode | | |
| species/strain | | |
| H. bacteriophora | Hb-GPS11 | Department of Entomology, Ohio |
| GPS11 | | state University, Wooster, Ohio, USA |
| H. bacteriophora | Hb-HP88 | Department of Entomology, Ohio |
| HP88 | | state University, Wooster, Ohio, USA |
| H. zealandica X1 | Hz-X1 | EcoGrow, Bondi Beach, NSW, |
| | | Australia |
| S. glaseri MB | Sg-MB | Becker Underwood Ltd, |
| | | Littlehampton, UK |
| S. glaseri NJ | Sg-NJ | Department of Entomology, Rutgers |
| | | University, New Brunswick, |
| | | NJ, USA |
| S. kraussei UK | Sk-UK | Becker Underwood Ltd, |
| | | Littlehampton, UK |
| Insecticide | | |
| Technical | | |
| name | | |
| Halofenozide | Mach2 2SC | Dow Agrosciences, Indianapolis, IN |
| Imidacloprid | Merit 75 WP | Bayer Corporation, Kansas City, MO |
| Trichlorfon | Dylox 6.2 G | Bayer Corporation, Kansas City, MO |

All the nematodes were reared in last instar Galleria mellonella L. following the methods described by Kaya and Stock (1997) for both the microplot and large field plot trials, except for H. zealandica which was mass-produced using a solid culture technique (Bedding, 1984) by EcoGrow, Australia and S. krausei which was produced by liquid fermentation technique by Becker Underwood Company, Littlehampton, UK. The nematodes reared in G. mellonella were stored in flat tissue culture flasks up to a maximum of 3 weeks prior to application in tap water at 10° C. at a concentration of 5-8×10³ IJs/ml. The commercially produced nematodes were stored for up to 1 week prior to the application in powdered formulations either at room temperature (H. zealandica) or at 5° C. (S. krausei). The second and third instar grubs used in these tests were either naturally occurring or artificially infested populations of P. japonica or C. borealis (see below).

Microplot Trials (Cylinder Studies)

Microplot trials consisted of field plots in which PVC cylinders, 20 cm inside diameter, were inserted to a depth of about 7 cm. Four microplot trials (Trials 14) were conducted on second instar P. japonica grubs. Treatments and environmental conditions at the time of nematode applications for each trial are summarized in Table 2. (Table 2 shows a summary of microplot trials on the efficacy of entomopathogenic nematodes and chemicals for the control of second instar Popillia japonica grub populations resulting from oviposition by the caged adults in Kentucky bluegrass lawns in Wooster or Columbus, Ohio during autumn 2000 to 2002 (Trials 1-4).)

TABLE 2

| | | | | | |
|---|---|---|---|---|---|
| | | | | Mean | |
| Treatment | Rate/ha^{a} | Applied | Observed | grubs/plot^{b} | % Control |

2000 - Wooster, Ohio
(Trial 1)

| | | | | | |
|---|---|---|---|---|---|
| H. bacteriophora HP88 | 2.5 × 10⁹ | Sept 13 | Oct 5 | 17.3 a | 52 |
| H. bacteriophora GSP11 | 2.5 × 10⁹ | Sept 13 | Oct 5 | 6.8 ab | 81 |
| H. zealandica X1 | 2.5 × 10⁹ | Sept 13 | Oct 5 | 9.8 ab | 73 |
| Mach 2 2SC (halofenzide) | 1.61 kg | July 13 | Oct 5 | 0.7 b | 98 |
| Untreated control | — | — | Oct 5 | 36.3 | — |
| 2001 - Wooster, Ohio | | | | | |
| (Trial 2) | | | | | |
| S. glaseri NJ | 2.5 × 10⁹ | Sept 12 | Oct 10 | 11.8 a | 20 |
| S. glaseri MB | 2.5 × 10⁹ | Sept 12 | Oct 10 | 8.8 ab | 41 |
| H. bacteriophora GPS11 | 2.5 × 10⁹ | Sept 12 | Oct 10 | 6.8 ab | 54 |
| H. zealandica X1 | 2.5 × 10⁹ | Sept 12 | Oct 10 | 3.0 bc | 80 |
| Imidacloprid (Merit 75 WP) | 0.34 kg | July 14 | Oct 10 | 0.3 d | 98 |
| Trichlorfon (Dylox 6.2G) | 9.0 kg | Sept 12 | Oct 10 | 1.3 cd | 92 |
| Untreated control | — | | | 14.8 a | — |
| 2001 - Columbus, Ohio | | | | | |
| (Trial 3) | | | | | |
| S. glaseri MB | 5.0 × 10⁹ | Sept 14 | Oct 9 | 6.4 ab | 26 |
| H. bacteriophora GPS11 | 5.0 × 10⁹ | Sept 14 | Oct 9 | 3.2 bc | 57 |
| H. zealandica X1 | 5.0 × 10⁹ | Sept 14 | Oct 9 | 2.4 c | 74 |
| Trichlorfon (Dylox 6.2G) | 9.0 kg | Sept 14 | Oct 9 | 6.4 ab | 29 |
| Untreated control | — | | Oct 9 | 7.6 a | — |
| 2002 - Wooster, Ohio | | | | | |
| (Trial 4) | | | | | |
| S. kraussei UK | 2.5 × 10⁹ | Sept 27 | Oct 18 | 15.2 a | 30 |
| H. bacteriophora GPS11 | 2.5 × 10⁹ | Sept 13 | Oct 18 | 0.8 b | 97 |
| H. zealandica X1 | 2.5 × 10⁹ | Sept 13 | Oct 18 | 0.5 b | 98 |
| Untreated | — | — | Oct 18 | 21.8 a | — |

^{a}Number of infective juveniles/ha or active ingredient/ha.
^{b}Means followed by the same letter are not significantly different at P > 0.05.

P. japonica adults were caged in the PVC cylinders to obtain grub populations following the methods described by Klein et al. (2000). Adult beetles were lured to Trece Catch Can traps (Trece Inc., Adair, Okla.) using a feeding lure containing PEG (phenol ethyl propionate, eugenol, and geraniol) and collected. Twice, at 1-week intervals, 30 adults consisting of approximately 50% females (measured volumetrically) were placed in each cylinder and covered with a nylon 20 mesh screen to induce egg laying within the cylinder area. The ovipositing beetles were fed fresh apple slices dusted with Brewer's yeast twice a week. Fourteen days after the initial caging, the nylon screens were removed. The developing grub population was monitored until the larvae reached the second instar, when all curative treatments were applied.

Trials 1, 2, and 4 were located at the Ohio Agricultural Research and Development Center, Wooster, Ohio whereas trial 3 was located at the TrueGreen Technical Center in Delaware, Ohio. Trials 1, 2, and 4 were set up on 1.5-m by 0.9-m plots, separated by 0.9 m alleyways, arranged in a complete randomized block design with four replications/treatment. For these trials, turf consisted of 70% Kentucky bluegrass and 30% dicot and monocot weed species, with level topography and no thatch was observed. Soil was a silt loam, with 2.8% OM, pH 6.0, 11.4 CEC. Trial 3 was conducted on Kentucky bluegrass lawn using 2.44 by 2.44 m plots arranged in a completely randomized block design with 5 replications/treatment. Soil was also a silt loam. Supplemental irrigation was applied prior to treatments to optimize beetle egg hatch and maintain actively growing turfgrass at both locations.

The nematodes were applied in 100 ml of water at 2.5×10⁹ IJs/ha as a curative treatment to each cylinder except for trial 3, in which they were applied at 5.0×10⁹ IJs/ha in 7.6 liters of water per plot with a hand held sprinkling can. Chemical insecticides were included for comparison in three of the trials. In trial 1, halofenozide was applied at 1.61 kg active ingredient (a.i.)/ha on Jul. 13, 2000 as a preventive treatment prior to P. japonica egg laying with a CO₂ sprayer and Teejet 8015 nozzles at 2.81 kg/cm² (40 psi) delivering 1630 liters/ha. In trial 2, imidacloprid (Merit 75 WP) was applied at 0.33 kg a.i./ha on July 14 and trichlorfon granules (Dylox 6.2G) at 9.0 kg a.i./ha with a shaker on September 14. In trial 3, trichlorofon was applied as in trial 2. All plots received post-application irrigation (see Table 2).

Large Field Plot Trials

Four field plot trials (Trials 5-8) were conducted on natural populations of P. japonica and C. borealis, or both species if they occurred together. Trial 5 was conducted on a naturally occurring mixed population of P. japonica and C. borealis grubs on a fairway at Wooldridge Woods Golf Course in Mansfield, Ohio in autumn 2001. Plots measuring 3-m by 3-m and separated by 0.9-m alleyways were arranged in a complete randomized block design with five replications/treatment. All treatments including imidacloprid (Merit 75WP) and trichlorfon (Dylox 6.2G) were applied on the morning of September 11 as curative treatments. Liquid applications were made with a sprinkling can at a volume of 8 liters/plot. Nematodes were applied at 2.5×10⁹ IJ/ha. The trichlorfon granules were applied using a Gandy Drop Spreader (Gandy Company, Owatonna, Minn.). Grub population consisted of 30% second and 70% third instar P. japonica, and 100% third instar C. borealis. Turf consisted of 70% Kentucky bluegrass and 30% annual bluegrass, topography level, height 2.5 cm, and had no thatch.

Trials 6 and 7 were conducted on stands of Kentucky bluegrass naturally infested with mixed populations of mostly third instar P. japonica and C. borealis in autumn 2001. The 1.2 by 1.2-m plots were located at the TrueGreen Technical Center in Delaware, Ohio. The nematodes were applied at 2.5×10⁹ IJs/ha in trial 6 and at 5.0×10⁹ IJ/ha in trial 7 with a CO₂ backpack sprayer with a 10 cm boom fitted with three 8005 LP flat fan nozzles at a volume of 813 liters/ha. Plots were arranged in a randomized complete block design with 5 replications/treatment for both trials.

Trial 8 was located on a lawn at the Ohio Agricultural Research and Development Center, Wooster, Ohio in autumn 2002. Plots measuring 1 m by 0.5 m separated by 0.9 m alleyways were arranged in a complete randomized block design with 3 treatments and 4 replications. At daybreak on September 13, all applications were made except for Sk-UK, which was applied on September 27 due to its late arrival. Nematodes were applied at 2.5×10⁹ IJs/ha using a hand held sprinkling can. Turf was 70% Kentucky bluegrass and 30% monocot and dicot weeds, height 7 cm, no thatch, and topography level. Soil was silt loam with 2.8% OM, pH 6.0, 11.4 CEC.

Efficacy data from all field trials were obtained from six, 18×18×7 cm deep samples removed from the middle of each plot. Soil from each square was lifted with a spade and placed in a plastic dishpan and the number of live grubs counted by hand sifting the soil and turf.

Statistical Analysis

Data on the total numbers of surviving grubs from each plot were subjected to the analysis of variance following the log₁₀ (X=1) transformation using Statsoft (1999) for each trial and means were separated by Tukey's mean separation test at P=0.05. In cylinder studies, the number of surviving grubs from all cylinders were pooled by plot. We also performed regression analysis to determine if the amount of post-application irrigation plus rainfall during the experiment had any influence on the effectiveness of the two most effective nematode strains. For predictability analysis, all data on the mean percentage control of all white grubs were pooled by treatment and subjected to the analysis of variance using Statsoft (1999) following arcsine transformation. Only trichlorfon was included in these analyses as it is applied as a curative treatment just like the nematodes.

Results

Microplot Trials

Wooster, Ohio, autumn, 2000 (Trial 1). Among the nematode species Hb-GPS11 applied September 13, provided the highest control of P. japonica (81%) as compared to the other treatments (Table 2). However, this level of control was not significantly different (P>0.05) from that obtained with Hb-HP88 (52%), Hz-X1 (73%) or halofenozide (98%). Halofenozide, applied July 13, was significantly more effective than Hb-HP88 (F=7.2; df=4, 18; P<0.002) but not from Hb-GPS11 or Hz-X1. Grub density in the control treatment on October 5 was high, at 383 grubs/m².

Wooster, Ohio, autumn 2001 (Trial 2). In this trial, the September 12 application of Hz-X1 provided 80% control of P. japonica and was not significantly less than trichlorfon, which provided 92% control (Table 2). Hb-GPS11 provided 54% control, which was not significantly different from Hz-X1 or the control, but was significantly lower than that from trichlorfon. Both S. glaseri strains provided poor control. Imidacloprid applied July 14 provided 98% control, which was significantly higher (F=9.64; df=6, 27; P<0.0001) than all nematode treatments but not significantly different from the September 12 trichlorfon treatment. Grub density in this trial on October 10 was average, at 152 grubs/m² in the control.

Columbus, Ohio, autumn 2001 (Trial 3). In this trial, Hz-X1 provided 74% control of P. japonica which was significantly higher (F=3.31; df=4, 12; P<0.034) than the 29% control from trichlorfon (Table 2). Hb-GPS11 again provided 57% control which was not significantly different from Hz-X1. Sg-MB was significantly less effective than Hz-X1. Grub density on October 9 in the control was low, at 78 grubs/m².

Wooster, Ohio, autumn 2002 (Trial 4). In this study, Hb-GPS11 and Hz-X1 applied September 13 provided 97 and 98% control of P. japonica and Sk-UK applied September 27 provided 30% control (Table 2). The grub density was significantly lower than the control (F=12.68; df=3, 12; P<0.002) only in the Hb-GPS11 and Hz-X1. The grub density in the control plots was high, at 225 grubs/m².

Large Field Plot Trials

Mixed Population of P. japonica and C. borealis on a Golf Course Fairway (Mansfield, Ohio, Autumn 2001; Trial 5)

Hz-X1 applied September 11 provided the highest control of natural populations of P. japonica (75%) as compared to all the other nematode and chemical treatments (Table 3). Only Hz-X1, Sg-MB, and trichlorfon were significantly more effective (F=5.88; df=5, 34; P<0.0001) when compared to the control against P. japonica. Although there were significant differences in the effectiveness of nematode treatments and chemical insecticides against C. borealis (Table 4), none of the treatments differed significantly from the control (F=1.78; df=5, 34; P<0.139). The Hz-X1 strain, Hb-GPS11 strain and trichlorfon were significantly more effective against C. borealis than the Sg-MB. Imidacloprid applied September 11 as a curative treatment had no effect on the survival of P. japonica, but caused a 47% reduction in C. borealis population. Density of P. japonica in the control was high, at 91 grubs/m² but C. borealis population was average at 38 grubs/m² with an overall density of both species at 129 grubs/m².

Table 3 shows the efficacy of entomopathogenic nematodes for the control of a mixed population of third instar Popillia japonica and Cyclocephala borealis grubs in a field plot trial on a fairway at Wooldridge Woods Golf Course, Mansfield, Ohio, during autumn 2001 (Trial 5).

TABLE 3

| | | P. japonica | | C. borealis | |
|---|---|---|---|---|---|
| | | Mean | | Mean | |
| | | grubs/ | % | grubs/ | % |
| Treament^{a} | Rate/ha | plot^{c} | Control | plot^{c} | control |
| S. glaseri MB | 2.5 × 10⁹ | 7.4 c | 58 | 10.0 a | 0 |
| H. BACTERIOPHORA | 2.5 × 10⁹ | 11.6 bc | 34 | 3.8 b | 47 |
| GPS11 | | | | | |
| H. zealandica X1 | 2.5 × 10⁹ | 4.4 c | 75 | 2.0 b | 72 |
| IMIDACLOPRID | 0.33 kg | 17.8 a | 0 | 3.8 ab | 47 |
| (MERIT 75 WP) | | | | | |
| Trichlorfon | 9.0 kg | 7.6 c | 57 | 3.8 b | 47 |
| (Dylox 6.2G) | | | | | |
| Untreated control | — | 17.2 ab | — | 7.2 ab | — |

^{a}Treatments applied on September 11 and data taken October 9 by counting the number of live grubs in six 18 × 18 × 7 cm deep samples from each plot.
^{b}Number of infective juveniles/ha or active ingredient/ha.
^{c}Means followed by the same letter are not significantly different at P > 0.05.

Mixed Population of P. japonica and C. borealis in a Kentucky Bluegrass Lawn (Columbus, Ohio, Autumn 2001; Trial 6)

In this trial, Hz-X1 and Sg-MB provided 66% and 74% control, respectively of the mixed population of P. japonica and C. borealis (Table 4) which was significantly higher (F=2.465; df=4, 16; P<0.044) than the 0% control from trichlorfon. Hb-GPS11 provided 41% control which was not significantly less than that by Hz-X1 or Sg-MB, but was not different from the control. Grub density on October 9 in the control was low at 56 grubs/m².

Nematode Concentration Study on a Mixed Population of P. japonica and C. borealis (Columbus, Ohio, Autumn 2001: Trial 7)

Efficacy of the two rates of the three nematode species against P. japonica or C. borealis did not differ significantly (P>0.05) when applied September 14 (Table 4). In this test, only Hz-X1 applied at the 2× rate (5×10⁹ IJs/ha) and trichlorfon applied at the standard rate provided significant control of P. japonica and C. borealis mixed population as compared to the control (F=2.96; df=7, 28; P<0.039). The level of grub control provided by all three nematode species at either rate was not significantly different (P>0.05) from that by trichlorfon, except for the low rate of Sg-MB. The grub density in the control plots was low at 84 grubs/m².

Table 4 shows efficacy of entomopathogenic nematode species and trichlorfon for the control of mixed populations of third instar Popillia japonica and Cyclocephala borealis grubs in field plot trials in a Kentucky bluegrass lawn at the TrueGreen Technical Center, Columbus, Ohio, during autumn 2001 (Trials 6 and 7).

TABLE 4

| Treatment^{a} | Rate/ha^{b} | Mean grubs/plot^{c} | % Control |
|---|---|---|---|
| TRIAL 6 | | | |
| S. glaseri MB | 2.5 × 10⁹ | 3.3 b | 74 |
| H. BACTERIOPHORA | 2.5 × 10⁹ | 7.0 ab | 41 |
| GPS11 | | | |
| H. zealandica X1 | 2.5 × 10⁹ | 2.7 b | 66 |
| Trichlorfon (Dylox 6.2G) | 9.0 kg/ha | 11.7 a | 0 |
| Untreated control | — | 10.0 a | — |
| Trial 7 | | | |
| S. glaseri MB | 2.5 × 10⁹ | 15.0 a | 6 |
| H. BACTERIOPHORA | 2.5 × 10⁹ | 10.3 ab | 36 |
| HP88 | | | |
| H. zealandica X1 | 2.5 × 10⁹ | 11.7 ab | 48 |
| S. glaseri MB | 5 × 10⁹ | 7.3 ab | 54 |
| H. BACTERIOPHORA | 5 × 10⁹ | 11.3 ab | 29 |
| HP88 | | | |
| H. zealandica X1 | 5 × 10⁹ | 2.7 b | 84 |
| Trichlorfon (Dylox 6.2G) | 9.0 kg | 4.0 b | 77 |
| Untreated control | — | 16.0 a | — |

^{a}Treatments applied on September 14 and data taken on October 9 by counting the number of live grubs in six 18 × 18 × 7 cm deep samples from each plot
^{b}Number of infective juveniles/ha or active ingredient/ha.
^{c}Means followed by the same letter are not significantly different at P > 0.05.

Third Instar C. borealis in a Kentucky Bluegrass Lawn (Wooster, Ohio. Autumn 2002: Trial 8)

The September 13 application of the nematodes was very effective with Hz-X1, Hb-GPS11 and Sk-UK providing 96, 83, and 50% control, respectively (Table 6). However, only the Hz-X1 and Hb-GPS11 treatments were significantly different (F=2.68; df=3, 15; P=0.024 ) from the control. The grub density in the control plots was very low at 31 grubs/m².

Table 5 shows efficacy of three entomopathogenic nematode species for the control of third instar Cyclocephala borealis grubs in a filed plot trial in a Kentucky bluegrass lawn at the Ohio Agricultural Research and Development Center, Ohio, during autumn 2002 (Trial 8).

TABLE 5

| | | | Mean | |
|---|---|---|---|---|
| | Date of | Date of | grubs/ | % |
| Treatment^{a} | application | observation | plot^{b} | Control |
| S. kraussei UK | Sept 27 | Oct 18 | 1.5 ab | 50 |
| H. BACTERIOPHORA | Sept 13 | Oct 18 | 0.5 b | 83 |
| GPS11 | | | | |
| H. zealandica X1 | Sept 13 | Oct 18 | 0.3 b | 96 |
| Untreated control | — | Oct 18 | 3.0 a | — |

^{a}Nematodes applied at 2.5 × 10⁹ infective juveniles/ha and data taken by counting the number of live grubs in five 18 × 18 × 7 cm deep samples from each plot
^{b}Means followed by the same letter are not significantly different at P > 0.05.

Relationship between the Amount of Post-Application Irrigation Plus Total Rainfall and Grub Control by the Nematodes

Polynomial regression model was a highly significant fit and described the relationship between nematode induced grub mortality and the amount of post-application irrigation plus the total rainfall during the experiment for Hb-GPS11 (R²_{adj}=0.85; P=0.003) and Hz-X1 (R²_{adj}=0.81; P=0.0009) strains. There was no significant relationship (P>0.05) between the amount of post-application irrigation plus rainfall and grub control in the trichlorfon treatment. At the optimum level of post-application irrigation plus rainfall, the grub control was 83-97% for Hb-GPS11 and 96-98% for Hz-X1 strains.

Predictability Analysis

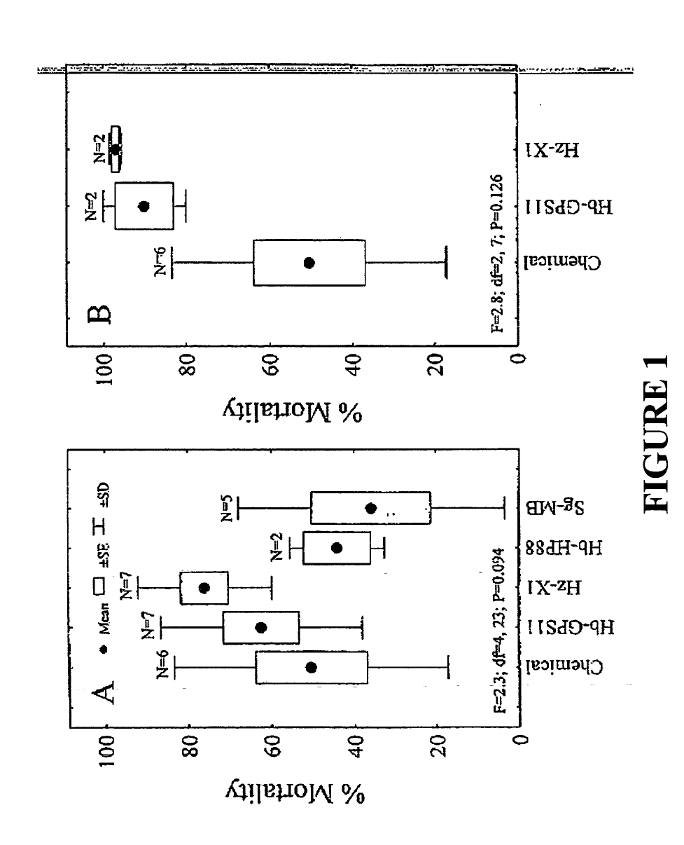

Combined analyses of pooled data across all trials in which the nematodes were applied at 2.5×10⁹ IJs/ha and trichlorfon was applied curatively at the standard rate indicated no significant differences (F=1.86; df=25; P=0.14) in the overall percent control provided by Hz-X1, Hb-GPS11, Hb-HP88, Sg-MB or trichlorfon (FIG. 1A). However, the variability in the level of control provided by the nematodes, especially Hz-X1, Hb-HP88 and Hb-GPS11 was smaller than that of the chemicals as indicated by the length of standard errors and standard deviations. Overall, the treatments for the white grub control ranked in descending order as follows: Hz-X1 (76.5±5.7%)>Hb-GPS11 (62.4±9.2%)>trichlorfon (50.33±13.5%)>Hb-HP88 (44±8.0%)>Sg-MB (35.8±14.4%).

Combined analyses of pooled data from all trials in which the post-application irrigation and precipitation were optimal for nematodes also did not reveal significant differences between the nematodes and trichlorfon (F=1.86; df=25; P=0.14) in the overall percent grub control. However, the mean grub control provided by the nematodes at optimum amount of post-application irrigation plus rainfall was almost twice as high as the trichlorfon treatment (FIG. 1B).

### Example 2

### Comparison of Virulence of GPS11 Strain to Other Nematode Strains on Various Grub Species

White Grubs

Three introduced grub species, P. japonica, A. orientalis and R. majalis and a native species C. borealis were used in this Example. Late instar grubs were collected from golf course rough areas and from nurseries in Ohio. The grubs were placed in dishpans containing field soil and stored at 5° C. up to a maximum of one month. Two days prior to the start of the experiments, the grubs were removed from cold storage and held at room temperature for acclimatization.

Nematodes

Nematode species and strains used in this study are listed in Table 6.

Nematodes were collected from either within or outside the current geographic ranges of the four white grub species studied. Heterorhabditis bacteriophora (strain HP88), H. indica Poinar, Karunakar & David (strain LN2), H. marelatus Liu & Berry (strain Oregon), H. megidis (strain UK), and H. zealandica Poinar (strain X1) were collected from outside the current geographic ranges of all four white grub species studied. In addition, H. bacteriophora (strains NC1 and Lewiston) were from areas where neither European chafer nor oriental beetle currently occurs.

TABLE 6

| | | | | |
|---|---|---|---|---|
| Species | Strain | Isolated | Original locality | Source |

Within the Range¹

| | | | | |
|---|---|---|---|---|
| H. bacteriophora | Oswego | 1990 | Oswego, New York, USA | Soil |
| H. bacteriophora | NC1 | 1975 | Clayton, North Carolina, USA | Heliothis zea |
| H. bacteriophora | Lewiston | 1993 | Lewiston, North Carolina, USA | Soil |
| H. bacteriophora | KMD10 | 1996 | Akron, Ohio, USA | Soil |
| H. bacteriophora | KMD19 | 1996 | Uniontown, Ohio, USA | Soil |
| H. bacteriophora | GPS1 | 1997 | Jeromesville, Ohio, USA | Soil |
| H. bacteriophora | GPS2 | 1997 | Jeromesville, Ohio, USA | Soil |
| H. bacteriophora | GPS3 | 1997 | Jeromesville, Ohio, USA | Soil |
| H. bacteriophora | GPS5 | 1997 | Jeromesville, Ohio, USA | Soil |
| H. bacteriophora | GPS11 | 1998 | Atwood, Ohio, USA | C. borealis |

Outside the Range²

| | | | | |
|---|---|---|---|---|
| H. bacteriophora | HP88 | 1982 | Logan, Utah, USA | Phyllophaga sp. |
| H. bacteriophora | Acows | 1994 | Ogallala, Nebraska, USA | Soil |
| H. indica | LN2 | 1992 | Coimbatore, India | Soil |
| H. marelatus | Oregon | 1996 | Seaside, Oregon, USA | Soil |
| H. megidis | UK | 1988 | England | Soil |
| H. zealandica | X1 | 1990 | New Zealand | Soil |

¹Nematodes isolated from areas overlapping with the current geographic ranges of Cyclocephala borealis and P. japonica.
²Nematodes isolated from areas outside the current geographic ranges of Cyclocephala borealis and P. japonica.

Virulence of Nematode Species/Strains to P. japonica and C. borealis

Virulence of H. indica, H. marelatus, H. megidis, H. zealandica, and 12 native strains of H. bacteriophora were evaluated against the introduced P. japonica and native C. borealis using the sand-based bioassay. Each last instar grub was exposed to 200 IJs in a 29.6 ml plastic portion cup (Comet Products Inc., Chelmsford, Mass.), with lids, containing 20 g heat-sterilized sand (particle size >170 μm) with 8% moisture content by weight (pF=1.3). A scarab larva was placed at the bottom of each cup prior to adding the sand. Nematodes were introduced in 0.5 ml of water into a centrally placed well (0.5 cm diameter, 1 cm deep) which was then filled with sand. Twenty grubs of each species were exposed to each nematode species or strain and additional 40 untreated grubs were used as controls. Grub mortality was recorded 2 weeks after incubation at 22° C. This experiment was repeated three times.

Nematode Dose Response Studies against Native and Non-Native Grub Species

Susceptibility of P. japonica, A. orientalis, R. majalis, and C. borealis to H. bacteriophora strain GPS11 (Ohio; see Example 1 above) and H. bacteriophora strain HP88 (Utah), H. megidis (UK), and H. zealandica (New Zealand) was compared over a range of nematode concentrations using a method described by Bedding et al. (1983). Last instar scarab larvae were individually exposed to different concentrations of nematodes in 148 ml plastic cups (Dart Container Corp., Mason, Mich.), with lids, filled with 80 g of heat-sterilized sand with 7% moisture content. There were five nematode concentrations each applied to 20 scarab larvae with a 40 untreated larvae as controls (total 140 larvae/nematode species). The nematode concentrations were 10, 33, 100, 330, and 1,000/grub. A scarab larva was placed at the bottom of each cup prior to adding the sand. Nematodes were introduced in 1 ml of water into a centrally placed well (0.5 cm diameter, 2 cm deep) which was then filled with sand. The cups were incubated at 22° C. Grub mortality was recorded 2 weeks after treatment. One grub species was tested at one time, and each experiment was repeated twice using different batches of nematodes.

Penetration Efficiency and Encapsulation of Nematodes in P. japonica and C. borealis

Penetration efficiency of H. bacteriophora, strains GPS11 and HP88, and H. zealandica strain X1 into P. japonica and C. borealis grubs was compared. Each last instar grub was exposed to 1,000 IJs of each nematode species/strain in 29.6 ml plastic portion cups (with lids) containing 20 g of heat-sterilized play sand with 8% moisture. Nematodes were introduced in 0.5 ml of water into a centrally placed well (0.5 cm diameter, 1 cm deep) which was then filled with sand. There were 15 replicates for each nematode species/strain for each scarab species (N=45). The cups were incubated at 25° C. All grubs were removed from the cups after 24 h, washed with distilled water to remove any nematodes on the body, and dissected to record the numbers of live, dead, and encapsulated nematodes. This experiment was repeated twice using different batches of nematodes.

Statistical Analyses

The grub mortality data in the dose response studies were analyzed using Probit analysis (Statsoft, 1999) and lethal concentrations for 30 and 50% mortality (LC₃₀ and LC₅₀) were determined. Significant differences among nematodes or grub species were based on the non-overlap of the 95% fiducial limits. Grub mortality data from other experiments were corrected for control mortality using Abbott's (1925) formula. Percentage data were normalized using arcsine transformation and subjected to analysis of variance (ANOVA) using Statsoft (1999). Differences among strains were compared using Tukey's mean separation test and linear contrasts using P=0.05. Hypotheses regarding the susceptibility of grub species or virulence of nematode strains collected from inside or outside the current geographic ranges of C. borealis and P. japonica were tested using contrasts. As the data from the experimental repeats yielded similar results, data from only the last set of experiments are presented.

Results

Virulence of Nematode Species/Strains to P. japonica and C. borealis

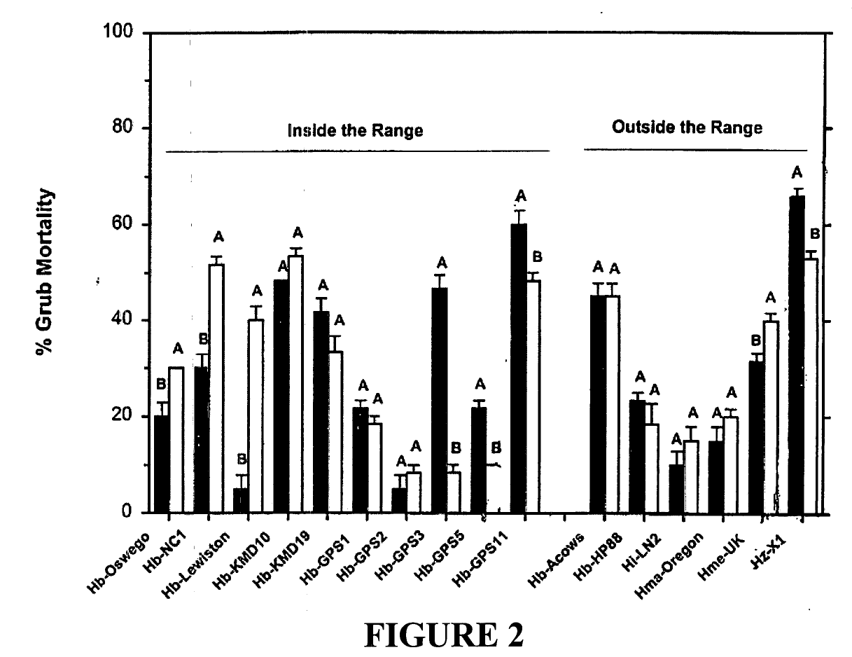

Susceptibility of P. japonica and C. borealis to different species and strains of Heterorhabditis differed significantly (F=53.06; df=15, 30; P<0.0001 for P. japonica and F=74.20; df=15, 30; P<0.0001 for C. borealis, respectively) (FIG. 2). Popillia japonica was significantly more susceptible (P<0.05) than C. borealis to GPS3, GPS5, and GPS11 strains of H. bacteriophora and X1 strain of H. zealandica, whereas C. borealis was more susceptible than P. japonica to Oswego, NC1, and Lewiston strains of H. bacteriophora and the UK strain of H. megidis (FIG. 2).

Overall, P. japonica was not significantly more susceptible to entomopathogenic nematodes considered as a group (P>0.05) than C. borealis (F=0.015; df=1; P=0.900). When considered as a group, the nematode species and strains from within and outside the geographic range of the two scarab species also did not differ in virulence significantly (F=3.57; df=1; P=0.068). Furthermore, there were no significant differences in the virulence of H. bacteriophora strains from outside the current geographic range of P. japonica and C. borealis as compared with those from within the range against any grub species (F=2.70; df=1; P=0.08 for P. japonica and F=0.44; df=1; P=0.50 for C. borealis, respectively). Interestingly, the virulence of H. bacteriophora strains, GPS1, GPS2, GPS3 and GPS5, isolated from the same golf course rough area differed significantly from each other against both grub species. The mean control mortality was 7.5% and 12.5% for P. japonica and C. borealis, respectively.

Nematode Dose Response Studies against a Native and Three Introduced Scarab Species

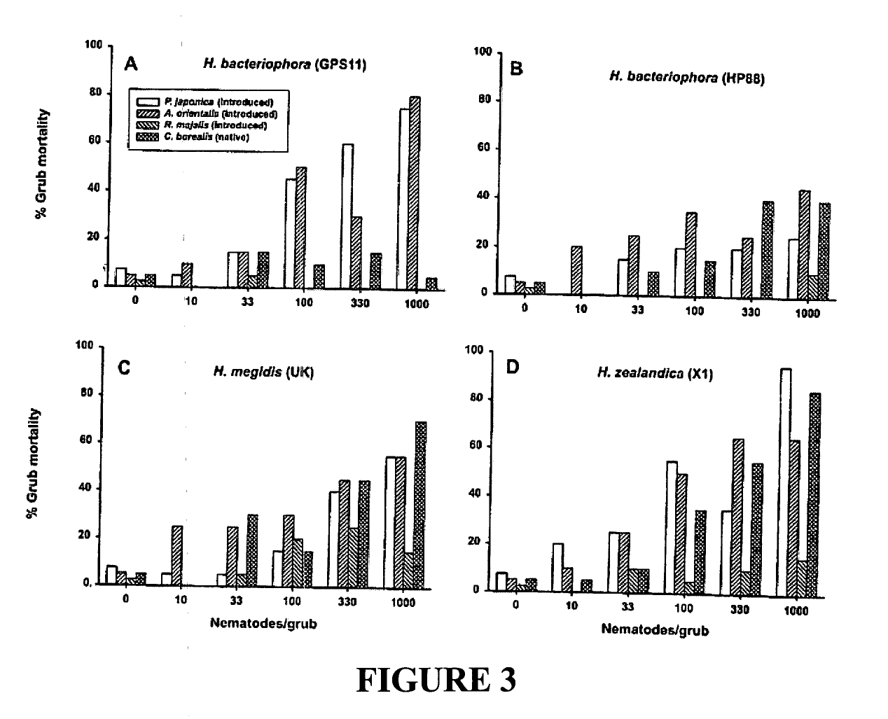

Susceptibility of the introduced A. orientalis, P. japonica, and R. majalis, and the native C. borealis to nematode species and strains differed considerably (FIG. 3A-D). The introduced R. majalis was the least susceptible to all the nematode species and strains tested. The highest mortality of this species was 27.5%, which was caused by the UK strain of H. megidis. The mortality of the other scarab species differed with nematode species, strain and concentration. For example, the indigenous H. bacteriophora strain GPS11 caused high mortality of A. orientalis and P. japonica, but produced less than 20% mortality of C. borealis (FIG. 3A). The control mortality in all the four scarab species was less than 8% in these experiments.

Heterorhabditis zealandica was significantly more virulent towards P. japonica than any other species or strain based on the non-overlap of the 95% fiducial limits for both the LC₃₀ and LC₅₀ values (Table 7). Heterorhabditis bacteriophora strain GPS11 was significantly more virulent than H. bacteriophora strain HP88 towards P. japonica based on LC₃₀ values. However, the two strains of H. bacteriophora and the UK strain of H. megidis did not differ significantly in virulence towards P. japonica when LC₅₀ values were compared. The virulence of nematode species and strains did not differ significantly against A. orientalis as the 95% fiducial limts overlapped for both LC₃₀ and LC₅₀ values (Table 7). Virulence of nematode species and strains did not differ significantly against C. borealis except for H. bacteriophora strain GPS11 for which the LC₃₀ and LC₅₀ values could not be estimated due to the low susceptibility of this scarab to the nematode (Table 7). (Table 7 shows mean numbers of nematodes required to cause 30% (LC₃₀) and 50% (LC₅₀) mortality of different white grub species¹ within 2 weeks. SE=Standard error; FL=Fiducial limits.)

TABLE 7

| | | | | | | |
|---|---|---|---|---|---|---|
| Treatment | LC₃₀ | SE | 95% FL | LC₅₀ | SE | 95% FL |

Popillia japonica

| | | | | | | |
|---|---|---|---|---|---|---|
| H. bacteriophora (GPS11) | 238 | 64 | 104-376 | 518 | 84 | 380-750 |
| H. bacteriophora (HP88) | 666 | 171 | 413-1398 | 1161 | 303 | 774-2655 |
| H. megidis (UK) | 493 | 91 | 332-732 | 817 | 136 | 612-1236 |
| H. zealandica (X1) | 104 | 28 | 46-161 | 272 | 36 | 211-361 |
| Anomala orientalis | | | | | | |
| H. bacteriophora (GPS11) | 234 | 66 | 97-376 | 520 | 88 | 378-766 |
| H. bacteriophora (HP88) | 381 | 115 | 162-733 | 860 | 210 | 576-1767 |
| H. megidis (UK) | 314 | 101 | 102-580 | 761 | 173 | 517-1433 |
| H. zealandica (X1) | 168 | 74 | 0-318 | 502 | 94 | 349-774 |
| Cyclocephala borealis | | | | | | |
| H. bacteriophora (GPS11) | —¹ | — | — | — | — | — |
| H. bacteriophora (HP88) | 216 | 42 | 149-359 | 345 | 69 | 249-614 |
| H. megidis (UK) | 147 | 33 | 85-242 | 282 | 57 | 202-498 |
| H. zealandica (X1) | 104 | 24 | 52-157 | 211 | 34 | 157-312 |

¹The LC₃₀ and and LC₅₀ values could not be determined for Rhizotrogus majalis due to its low susceptibility to all the nematodes and for C. borealis due to its low susceptibility to H. bacteriophora strain GPS11.

Interestingly, the non-indigenous H. zealandica was significantly more virulent towards the native C. borealis than the introduced A. orientalis based on the non-overlap of 95% fiducial limits for the LC₅₀ values (Table 7). Also the non-indigenous H. megidis (UK strain) was significantly more virulent towards the native C. borealis than the introduced A. orientalis and P. japonica. The HP88 strain of H. bacteriophora, originally isolated from outside the geographic ranges of P. japonica and C. borealis was significantly more virulent towards the native C. borealis than the introduced P. japonica based on both the LC₃₀ and LC₅₀ values. Virulence of none of the nematode species or strains differed significantly against P. japonica and A. orientalis (Table 7).

Penetration Efficiency and Encapsulation of Nematodes by P. japonica

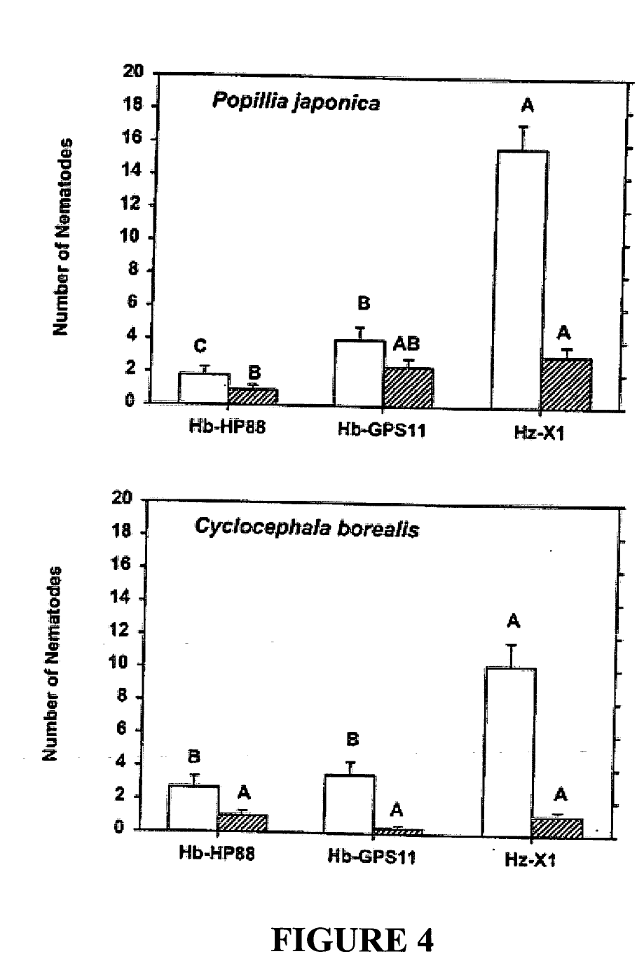

Number of nematodes that penetrated into the late instar P. japonica and C. borealis within 24 h after exposure differed significantly among the nematode species and strains (F=42; df=2, 42; P<0.0001 for P. japonica and F=15.8; df=2, 42; P<0.0001 for C. borealis, respectively) (FIG. 4). H. zealandica had the highest penetration, followed by H. bacteriophora GPS11, and H. bacteriophora HP88 in both scarab species. The overall nematode penetration was very low, only 0.2, 0.4, and 1.6% of H. bacteriophora HP88, H. bacteriophora GPS11, and H. zealandica penetrated P. japonica within 24 h, respectively. Penetration into C. borealis grubs was only 0.3, 0.4, and 1.0% for H. bacteriophora HP88, H. bacteriophora GPS11, and H. zealandica, respectively.

The three strains also differed significantly in the numbers of nematodes that were killed due to encapsulation by P. japonica (F=33; df=2, 42; P<0.0001) but not by C. borealis (P>0.05; FIG. 4). An average of 21, 50, and 56% of H. zealandica, H. bacteriophora HP88, and H. bacteriophora GPS11, respectively, were found to be dead and melanized due to encapsulation within 24 h in P. japonica. The numbers of nematodes encapsulated and melanized in C. borealis was much less: 10, 11, and 40% of H. bacteriophora GPS11, H. zealandica, and H. bacteriophora HP88, respectively, were killed due to encapsulation by C. borealis (FIG. 4).

### Example 3

### Longevity and Stress Resistance

Nematode Populations

Fifteen populations of H. bacteriophora collected from diverse locations were used (see Table 8). These populations were confirmed to be H. bacteriophora using dauer juvenile length, male tail morphology, and isozyme analyses (Jagdale and Grewal, unpublished data). The enzyme systems used were: arginine kinase, fumrate hydratase, glycerol-3-phosphate dehydrogenase, isocitrate dehydrogenase, mannose-6-phosphate isomerase, malate dehydrogenase, phosphoglucomutase, and phosphoglucoisomerase. Seven of these populations were collected from Ohio and were maintained in the laboratory for 4-14 months and were cultured only two to four times in the wax moth, Galleria mellonella (Lepidoptera: Pyralidae) larvae during this period. Four (GPS1, GPS2, GPS3, and GPS5) of these seven populations were collected from a golf course rough area (termed grassland) of approximately 200 m² in Jeromesville, Ohio. Two populations (Lewiston-IBCS and HP88-MRD) were obtained from commercial producers of entomopathogenic nematodes and have been derived from the Lewiston and HP88 populations originally isolated from North Carolina and Utah, respectively. Other populations were obtained from different researchers who have maintained them using similar culture methods for different periods (see Table 8). All nematode populations used in this study have been isolated from the soil using G. mellonella larvae as a bait except HP88, GPS11, and NCI which were isolated from naturally infected insects collected from the field. All populations were cultured three times in the laboratory in the wax moth larvae at 25° C. using the methods described by Kaya and Stock (1997) prior to the initiation of this study in 1998.

TABLE 8

| | | | | Average |
|---|---|---|---|---|
| | | | Average | annual |
| | | | maximum annual | rainfall |
| Population | Isolated | Original locality | temperature (° C.) | (cm) |
| NC1 | 1975 | Clayton, North | 22.17 | 115.57 |
| | | Carolina | | |
| HP88 | 1982 | Logan, Utah | 16.33 | 45.03 |
| HP88-MRD | 1982 | Logan, Utah | 16.33 | 45.03 |
| Riwaka | 1990 | Riwaka, | 17.26 | 276.23 |
| | | New Zealand | | |
| Oswego | 1990 | Oswego, New York | −6.56 | 127.97 |
| Lewiston- | 1993 | Lewiston, North | 22.22 | 117.32 |
| IBCS | | Carolina | | |
| OH25 | 1993 | Hermiston, Oregon | 18.17 | 22.76 |
| Acows | 1994 | Ogallala, Nebraska | 17.39 | 47.57 |
| KMD10 | 1996 | Akron, Ohio | 15.06 | 93.12 |
| KMD19 | 1996 | Union town, Ohio | 15.06 | 93.98 |
| GPS1 | 1997 | Jeromesville, Ohio | 14.94 | 98.93 |
| GPS2 | 1997 | Jeromesville, Ohio | 14.94 | 98.93 |
| GPS3 | 1997 | Jeromesville, Ohio | 14.94 | 98.93 |
| GPS5 | 1997 | Jeromesville, Ohio | 14.94 | 98.93 |
| GPS11 | 1998 | Atwood, Ohio | 15.72 | 83.72 |

Longevity

Longevity of the dauer juveniles of the 15 populations was assessed at 25° C. in water in 24-well plates (Falcon No. 3047). Two thousand dauer juveniles were placed in each well in 1-ml of autoclaved tap water and all 24-wells were used for one population. The plates were sealed with parafilm to minimise evaporation. The number of live and dead nematodes were counted by taking three, 50 μl-drops from each well after carefully shaking its contents, once a week, for 9 weeks, and percent survival was calculated. A 150 μl autoclaved tap water was added to the well after the removal of the samples. Four randomly selected wells were used at each sampling time. The inactive nematodes were considered dead if they did not respond to prodding with a steel probe. The experiment was repeated twice.

Stress Tolerance

Heat. Heat tolerance of dauer juveniles of different populations was assessed by exposure to 40° C. for 2 h. Five thousand dauer juveniles were placed in a 5-cm diameter petri dish in 5-ml of water. Six replicates were prepared for each population. After exposure to 40° C., the dishes were incubated at 25° C. for 24 h for recovery of the nematodes from heat-shock. Six additional dishes constantly maintained at 25° C. for each population were used as control. Nematode survival was determined by taking three, 50 μl samples from each plate, as described above. The experiment was repeated three times.

Hypoxia. Tolerance to hypoxia was examined by storing the dauer juveniles in 0.5 ml of water in 0.5 ml Eppendorf tubes. Ten thousand dauer juveniles were held in each tube at 25° C. with the lids tightly closed. Previous experiments using similar concentrations of H. bacteriophora dauer juveniles in glass bottles showed that the dissolved oxygen reaches zero within 10 min after the bottles were closed (P. S. Grewal, unpublished results). Five tubes were prepared for each population. After 96 h, the nematodes were transferred into 10-cm diameter petri dishes containing 9 ml of water and were incubated at room temperature for 24 h for the recovery of the nematodes from hypoxia. Five, 5-cm diameter dishes containing nematodes at the same concentration were used as control and were held without hypoxia at room temperature. Nematode survival was then determined as above. The experiment was repeated three times.

Desiccation. Desiccation tolerance of the nematode populations was evaluated by dehydrating the dauer juveniles in glycerol solution as described by Glazer and Salame (2000). Five thousand dauer juveniles in 0.5 ml water were mixed in 0.5 ml of 50% glycerol solution to obtain a final concentration of 25%. The nematodes mixed in glycerol were placed in 24-well plates with six wells for each population. After incubation at 25° C. for 72 h, the nematodes were rehydrated in 10 ml of water. Six additional wells containing nematodes in water at the same concentration were used as control for each population. Nematode survival was determined 24 h after rehydration in water at 25° C. The experiment was repeated three times.

UV. Tolerance of dauer juveniles to UV was assessed using the methods described by Gaugler et al. (1992) with some modifications. Ten thousand dauer juveniles in 2-ml of water in 5-ml petri dishes, with lids removed, were exposed to a UV lamp (Model EB-280, Spectronics Corp.) that emitted a medium wave UV (average 302 nm) for 5 min. The virulence of the irradiated and non-irradiated dauer juveniles to the wax moth larvae was then determined following the five-on-one sand-well bioassay described by Grewal et al. (1999). Five dauer juveniles in 100 μl of water were introduced in each well of a 24-well plate containing 1.5 g dry play-sand (Lonestar No. 60, Lapis Lustre <710 μm) per well. One last-instar G. mellonella larva was placed in each well and the plates were incubated at 25° C. Four well plates were set up as replicates for each population, with 12 wells for irradiated nematodes and 12 for non-irradiated nematodes (N=48 per treatment). Percent larval mortality was recorded 6 days after inoculation. The experiment was repeated three times. The percent of original virulence remaining after exposure to UV was calculated using the following equation:

$$\% Original Virulence Remaining = \frac{\% larval mortality by irradiated nematodes}{\% larval mortality by non \text{-} irradiated nematodes} \times 100$$

Statistical Analyses

Percent data were normalized using arcsine transformation and subjected to analysis of variance (ANOVA). Differences among populations were compared using Tukey's multiple range test (MINITAB, Minitab, Inc., 1996). Longevity was defined as either LT₅₀ (weeks to 50% mortality) or LT₉₀ (weeks to 90% mortality) and was calculated by Probit analysis module of the Maximum Likelihood Program (National Algorithms Group, UK, 1987). Pearson's correlation and Kendall Rank correlation analyses were used to determine the relationship between longevity and tolerance to various stress factors and to determine relationships between different stress factors. Arcsine transformed survival data were used in Pearson correlation, but not in Kendall rank correlation analysis. We also used Kendall rank partial correlation to determine the relationship between the positively correlated stress factors independent of their relationship with longevity, i.e., keeping longevity constant (Siegel, 1956).

The correlation matrix of the five variables was also analyzed by principal components analysis (PCA). In a PCA the structure of a set of interrelated variables is analyzed by fitting mutually orthogonal (i.e., noncorrelated) factors to the correlation matrix. Factors are fitted until a desired proportion of the variance has been accounted for. These factors extract the variance that is shared by one or more variables in the correlation matrix. Thus, the first factor removes the most variance and succeeding factors remove progressively less additive variance from the correlation matrix. By plotting the contributions of each variable to the factors in a 3 dimensional plot, it is possible to deduce relationships between the variables. Correlation analysis was also used to assess relationship of dauer juvenile longevity and stress tolerance with period of laboratory culture and rainfall or temperature patterns of the original localities of the populations. All comparisons were made at the 0.05% significance level. Back-transformed data are presented in the text and figures.

Results

Longevity

The dauer juvenile longevity, as defined by the LT₅₀ₛ and LT₉₀ₛ varied between 4 and 11 weeks and 6 and 16 weeks, respectively among the populations (see Table 9, which shows estimated time (in weeks) to 90% (LT₉₀) and 50% (LT₅₀) mortality of dauer juveniles of different populations of H. bacteriophora with standard errors (S.E.) and 95% confidence intervals (C. I.).). Two populations, Lewiston-IBCS and HP88-MRD ranked as the shortest lived populations with LT₉₀ of only 6.4 and 6.8 weeks, respectively, and KMD19 ranked as the longest lived population with LT₉₀ of 16 weeks. Dauer juvenile longevity differed significantly among several populations based on the non-overlap of confidence intervals. Dauer longevity also differed significantly among populations isolated from a single grassland locality (golf course) in Ohio (GPS1, GPS2, GPS3, and GPS5) and varied between 10.6 and 14.6 weeks. The overall mean life span (LT₉₀) of the dauer juveniles for all H. bacteriophora populations was about 11 weeks. Longevity of the populations showed no significant (P>0.05) relationship with the number of years of maintenance in the laboratory after isolation from the field.

TABLE 9

| Population | LT₉₀ | S.E. | 95% C.I. | LT₅₀ | S.E | 95% C.I. |
|---|---|---|---|---|---|---|
| NC1 | 10.941 | 0.174 | 10.621-11.306 | 7.639 | 0.075 | 7.498-7.793 |
| HP88 | 8.279 | 0.131 | 8.033-8.550 | 4.990 | 0.058 | 4.878-5.106 |
| HP88-MRD | 6.779 | 0.095 | 6.603-6.976 | 4.281 | 0.045 | 4.192-4.369 |
| Riwaka | 10.219 | 0.161 | 9.925-10.558 | 7.473 | 0.074 | 7.333-7.626 |
| Oswego | 10.271 | 0.144 | 10.003-10.571 | 7.008 | 0.065 | 6.885-7.138 |
| Lewiston-IBCS | 6.417 | 0.074 | 6.278-6.572 | 4.459 | 0.035 | 4.391-4.529 |
| OH25 | 12.207 | 0.225 | 11.802-12.692 | 9.599 | 0.111 | 9.398-9.835 |
| Acows | 12.439 | 0.216 | 12.046-12.898 | 9.336 | 0.012 | 9.148-9.550 |
| KMD10 | 12.240 | 0.241 | 11.807-12.759 | 9.361 | 0.116 | 9.148-9.607 |
| KMD19 | 16.102 | 0.647 | 14.976-17.555 | 11.122 | 0.333 | 10.535-11.865 |
| GPS1 | 14.601 | 0.377 | 13.923-15.416 | 10.192 | 0.180 | 9.865-10.576 |
| GPS2 | 11.437 | 0.185 | 11.097-11.827 | 8.076 | 0.083 | 7.920-8.248 |
| GPS3 | 10.656 | 0.148 | 10.382-10.966 | 7.591 | 0.068 | 7.462-7.728 |
| GPS5 | 11.323 | 0.169 | 11.010-11.678 | 8.064 | 0.075 | 7.923-8.218 |
| GPS11 | 12.668 | 0.239 | 12-233-13.178 | 9.381 | 0.113 | 9.172-9.619 |

Stress Tolerance

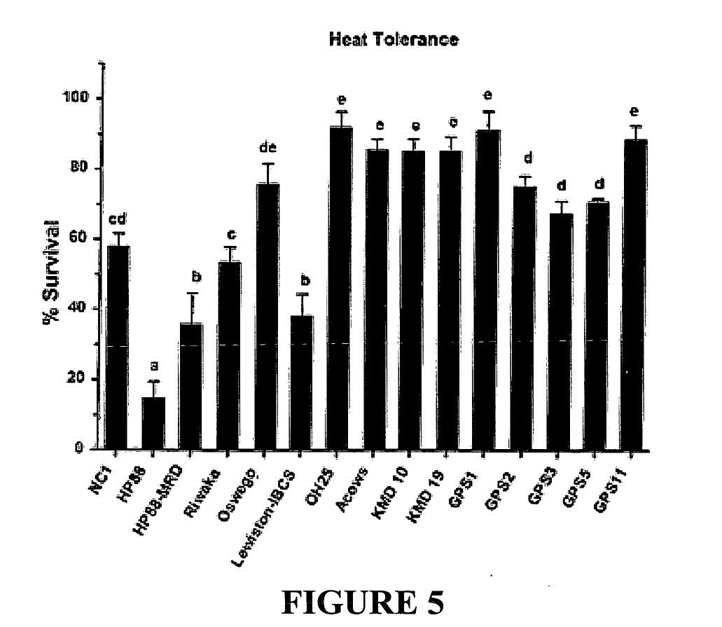

Heat. Populations of H. bacteriophora differed significantly (F=57.5, df=14, P<0.05) in heat tolerance (FIG. 5). Survival of dauer juveniles exposed to 40° C. for 2 h ranged between 16 and 92% among the populations with OH25 showing maximum survival and HP88 showing the least. Six populations had over 80% survival and three had less than 40%. Among the four populations isolated from the same grassland locality, one (GPS1) was significantly more heat tolerant than the other three (GPS2, GPS3, and GPS5). There was no significant (P>0.05) relationship between heat tolerance of populations and the number of years of maintenance in the laboratory after isolation from the field or the average maximum temperature of the original localities of the populations.

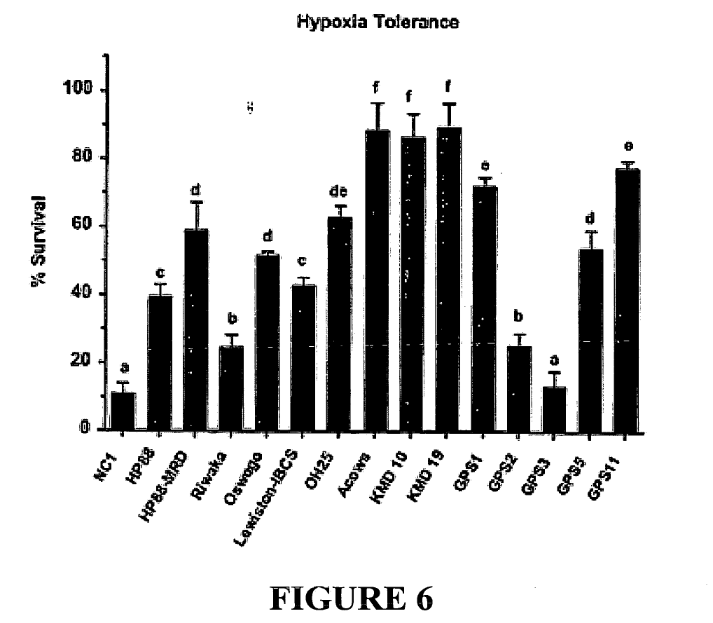

Hypoxia. Survival of H. bacteriophora populations under hypoxic conditions also differed significantly (F=98.8, df=14, P<0.05) (FIG. 6). Survival following exposure to hypoxic conditions for 96 h varied between 10 and 90% among the populations with KMD19 showing maximum survival and NC1 possessing the lowest survival. Three populations (Acows, KMD10 and KMD19) had over 80% survival, two (NC1 and GPS3) had less than 20% survival, and two (GPS2 and Riwaka) had between 20-40% survival. Again significant differences were observed in hypoxia tolerance among populations isolated from a grassland locality (i.e., GPS1, GPS2, GPS3, and GPS5). There was no significant (P>0.05) relationship between hypoxia tolerance of populations and the number of years of maintenance in the laboratory after isolation from the field or the average annual rainfall of the original localities of the populations.

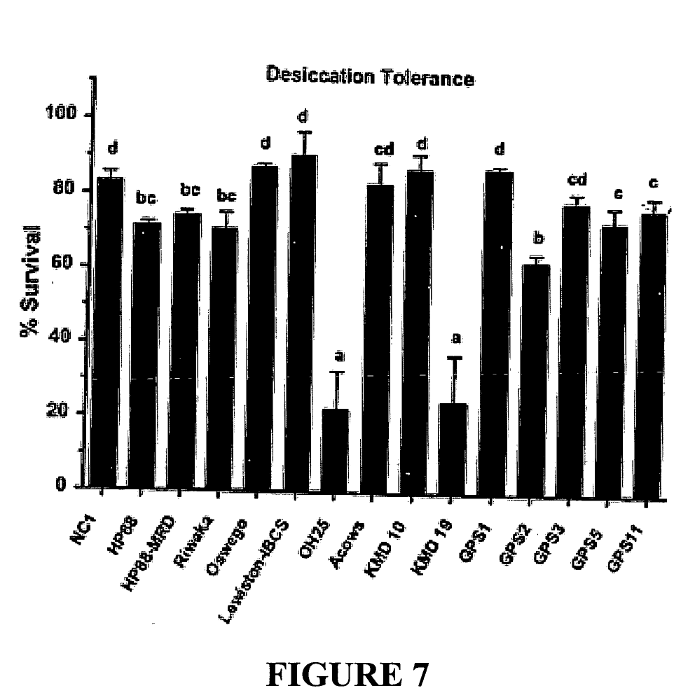

Desiccation. Desiccation survival also differed significantly (F=24.9, df=14, P<0.05) among 15 populations of H. bacteriophora (FIG. 7). Survival of dauer juveniles following osmotic desiccation (water activity, Aw=0.944) for 72 h varied between 23% and 95% among the 15 populations. Seven populations had over 80% survival, but two (OH25 and KMD19) had only about 20% survival. Desiccation tolerance was also significantly different among the populations (i.e., GPS1, GPS2, GPS3, and GPS5) isolated from one grassland locality. There was no significant (P>0.05) relationship between desiccation tolerance of populations and the number of years of maintenance in the laboratory after isolation from the field or the average annual rainfall of the original localities of the populations.

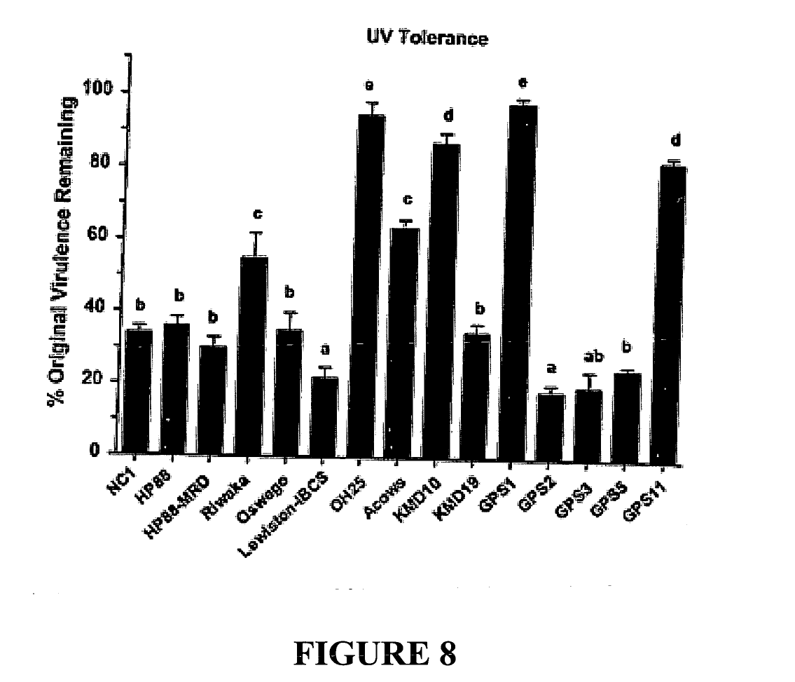

UV. Tolerance to UV, determined on the basis of the original virulence retained by the dauer juveniles following exposure to medium wave UV for 5 min, also differed among the 15 populations of H. bacteriophora (F=11.1, df=14, P<0.05). Virulence declined in all populations after exposure to UV (FIG. 8). Two populations (GPS1 and OH25) maintained over 95% of the original virulence, two populations (GPS11 and KMD10) maintained between 85-88%, and three populations (GPS2, GPS3, and Lewiston-IBCS) had retained only less than 20% of the original virulence. Further, the populations isolated from the same grassland locality (i.e., GPS1, GPS2, GPS3, and GPS5) also differed significantly for UV tolerance. There was no significant (P>0.05) relationship between UV tolerance of populations and the number of years of maintenance in the laboratory after isolation from the field.

Relationships between Longevity and Stress Tolerance and Between Different Stressors

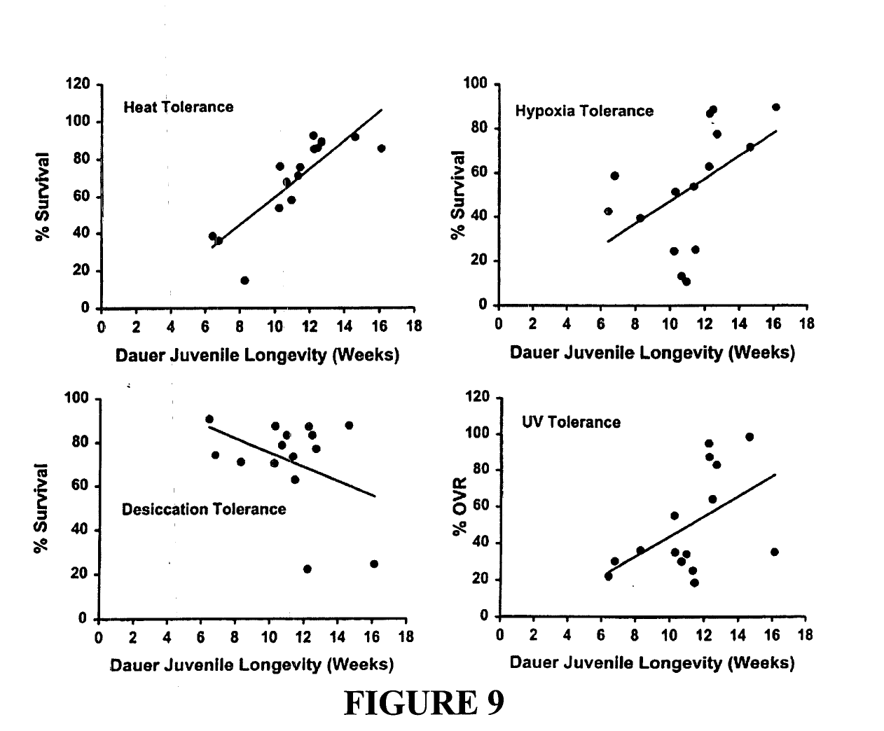

As the relationships between dauer juvenile longevity and tolerance to different stresses were similar when longevity was defined as LT₅₀ or LT₉₀, only the LT₉₀ data are presented in FIG. 9. Dauer juvenile longevity was positively correlated with heat, UV, and hypoxia tolerance, but not with desiccation tolerance (FIG. 9). Longevity showed the strongest positive correlation with heat tolerance in both the Pearson correlation and Kendall rank correlation analyses (Table 10). Relationship between longevity and UV, and longevity and hypoxia was only found to be positively correlated using the Kendall rank correlation analysis (Table 10). An assessment of the relationships between different stressors, revealed that only UV was positively correlated with heat and hypoxia and this relationship was significant in both Pearson and Kendall rank correlation analyses (Table 10). Kendall partial rank correlation analysis revealed that the correlation coefficients between heat and UV and hypoxia and UV decreased when longevity was held constant, indicating that their mutual correlations were partly due to their individual correlation with longevity. The Kendall rank partial correlation coefficients between heat and UV and hypoxia and UV, were 0.376 and 0.268 as compared to Kendall rank correlation coefficients of 0.485 and 0.381, respectively.

TABLE 10

| | | | Kendall | |
|---|---|---|---|---|
| | Pearson | | Rank | |
| | correlation | | correlation | |
| Parameter | coefficient | p | coefficient | p |
| Longevity and heat | 0.759 | 0.001 | 0.685 | 0.0001 |
| Longevity and UV | 0.504 | 0.055 | 0.362 | 0.030 |
| Longevity and hypoxia | 0.497 | 0.059 | 0.447 | 0.009 |
| Longevity and desiccation | −0.397 | 0.143 | −0.057 | 0.382 |
| Heat and UV | 0.610 | 0.015 | 0.485 | 0.005 |
| Heat and Hypoxia | 0.394 | 0.146 | 0.267 | 0.082 |
| Heat and desiccation | −0.245 | 0.379 | 0.048 | 0.401 |
| Hypoxia and desiccation | −0.181 | 0.518 | 0.286 | 0.068 |
| Hypoxia and UV | 0.551 | 0.033 | 0.381 | 0.024 |
| UV and desiccation | −0.085 | 0.763 | 0.057 | 0.382 |

The first three principal components accounted for almost 90% of the variation in the correlation matrix of longevity and the four stress factors (see Table 11, which shows Eigenvalues of the correlation matrix relating the five variables, longevity, resistance to heat, hypoxia, UV, and desiccation).

TABLE 11

| | | Cumulative | % Variance | Cumulative % |
|---|---|---|---|---|
| Axis | Eigenvalue | Eigenvalue | accounted for | variance |
| 1 | 2.920313 | 2.920313 | 58.40627 | 58.40627 |
| 2 | 0.997483 | 3.917796 | 19.94965 | 78.35592 |
| 3 | 0.574896 | 4.492692 | 11.49791 | 89.85384 |
| 4 | 0.441039 | 4.933731 | 8.82077 | 98.67461 |

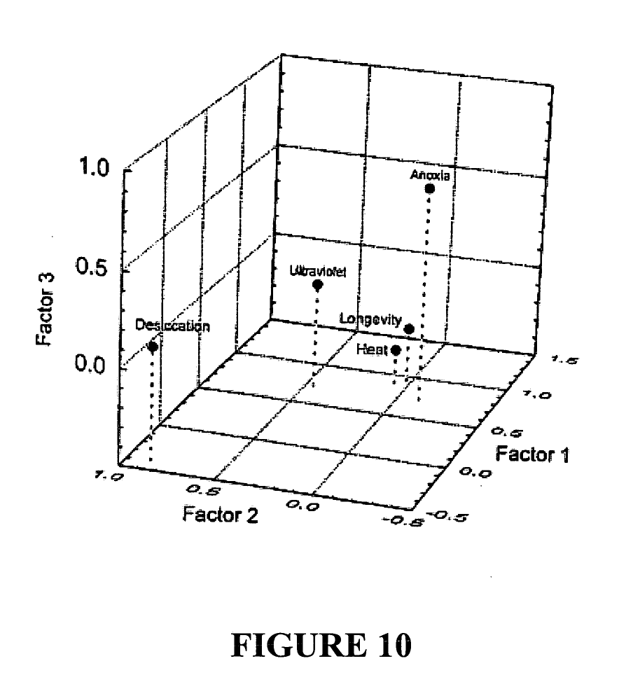

Analysis confirmed the strong positive correlation between longevity and heat tolerance; the Eigenvalues for Factors 1, 2, and 3 accounted for nearly identical variance for longevity and heat tolerance (FIG. 10; Table 12, which shows identification of the principal components). Closer examination of the first three factors in FIG. 10 shows also that longevity and tolerance to heat and hypoxia are clustered in Factors 1 and 2 and differ in Factor 3. Longevity and tolerance to heat and UV have similar contributions to Factors 1 and 3. By contrast, desiccation has quite different values for all of the first three factors, suggesting the absence of a relationship of this variable with the others.

TABLE 12

| | 1 Factor | 2 Factors | 3 Factors | 4 Factors | Multiple R² |
|---|---|---|---|---|---|
| Longevity | .899500 | .900260 | .944428 | .961911 | .888923 |
| Heat | .806260 | .812277 | .912748 | .973072 | .853936 |
| Hypoxia | .536977 | .549876 | .968917 | .999702 | .362995 |
| UV | .523543 | .733302 | .736952 | .999403 | .434548 |
| Desiccation | .154034 | .922081 | .929647 | .999642 | .202716 |

### Example 4

### Use of GPS11 Nematodes for Control of Black Vine Weevil (BVW)

There are currently no known curative controls for the black vine weevil (BVW), Otiorhynchus sulcatus (Coleoptera: Curculionadea) larvae in container nursery stock. This Example is designed to show the ability of GPS11 to control the BVW.

Two-hundred potted Astilbe, Astilbe astilboides (Spiraea: Rosaceae) in one-gallon containers infested with overwintered BVW larvae from a nursery in Lake County, Ohio. The pots were transported to greenhouses at the Ohio Agricultural Research and Development Center. Treatments were completely randomized and replicated four times. To ameliorate variation in BVW numbers between pots, three randomly selected and grouped pots constituted a plot. Treatments were applied evenings on April 17 and 18 to the pots in a 300-ml drench per pot. After all treatments were applied, a light syringing was done to wash the nematodes into the soil.

Six species of entomopathogenic nematodes were compared in this Example. Heterorhabditis bacteriophora strains HP88 and GPS11, H. marelatus, H. megidis, H. indica, Steinernema carpocapsae, and S. feltiae were all applied at 2.5 billion/hectare based on the surface area of the container. Data taken 16 days after treatment (DAT by destructive sampling of the plants and growing media in each pot. The number of live BVW larvae and pupae were recorded. Total from the three pots constituting a replicate were combined for an analysis of variance and means separated by LSD test at p=0.05.

Results

All nematode treatments except S. feltiae provided significant control (Table 13). Both of the Steinernematids had an average of 2 BVW per pot. All of the Heterorhabditids gave excellent control, of 90% or greater. H. bacteriophora strain HP88 eradicated the BVW from the pots and GPS11 provided near-eradication, at 98% reduction.

TABLE 13

| | Black Vine Weevils Larvae and Pupae 16 DAT^{B} | | | |
|---|---|---|---|---|
| Treatment^{A} | Total | Mean/Rep^{c} | Mean/Plant | % Reduction |
| H. bacteriophora | 0 | 0.00e | 0 | 100 |
| HP88 | | | | |
| H. bacteriophora | 2 | 0.50de | 0.2 | 98 |
| GPS11 | | | | |
| H. marelatus | 8 | 2.00cde | 0.7 | 93 |
| H. megidis | 1 | 0.25de | 0.1 | 99 |
| H. indica | 12 | 3.00cd | 1.0 | 90 |
| S. carpocapsae | 24 | 6.00bc | 2.0 | 79 |
| S. feltiae | 39 | 9.75ab | 3.3 | 67 |
| Water - untreated | 46 | 11.5a | 9.9 | — |

^{A}Applied April 17, in 300 ml/pot. Each treatment replicated 4 × 3 pots each.
^{B}Data based on the total live weevil larvae in twelve 15-cm diameter pots.
^{C}Data transformed to log10 (X + 1) for analysis. Means followed by the same letter are not significantly different.

### Example 5

### Use of GPS11 Nematode in Controlling the Grape Root Borer

Grape root borer eggs were obtained by collecting pupae from a field in North Carolina, and held in the laboratory until adult emergence, at which time females were placed with males for mating. Mated females were placed in individual containers until egg-laying activity was completed and the females had expired. Fertile eggs were collected and placed in small glass vials for shipment to Wooster, Ohio.

Upon arrival in Ohio, the eggs were placed on filter paper placed in the bottom of a 450 ml holding plastic container. Holding cages were equipped with solid lids to minimize moisture loss. Approximately 1 ml of water was added to each holding container to moisten the filter paper. Seven containers were maintained in this manner. Containers were checked daily for newly emerged GRB larvae and water was added when the filter paper became dry. Neonate larvae were removed from the holding containers daily and placed on sections of grape root. A small, artist's paintbrush was used for transferring the larvae. Two-year-old pot-grown “Concord” vines were the source of root material used for rearing GRB larvae.

Rearing newly hatched GRB larvae was accomplished by placing two sections of grape root (ca. 3-5 mm in diameter and approximately 2 cm in length) in the bottom of a 30-ml milk creamer container, along with a 1.5-cm² piece of blotter paper. The blotter paper was placed in the bottom of the container prior to adding the root sections and water was added to the paper to maintain moisture. Two newly hatched larvae were then placed on the root pieces and the container was sealed with a plastic lid.

Our preliminary work in 1998 showed about 50% survival for the young larvae in this excised root system. The rearing containers were maintained in the lab at 22° C. for about a month before being exposed to the nematodes. Additional moisture was added to the containers when the blotter paper became visibly dry. Prior to treatment with nematodes, 10 g of sterilized dry sand (Lonestar No. 60, Lapis Lustre >710 μm) was added to each rearing container covering the root pieces. Five hundred nematodes were then added to each container and water was added to bring the moisture level of the sand to 8%. Seventeen nematode species and strains (Table 1) were screened for their ability to infect first year GRB larvae. Detailed information on the original locality and year of isolation of all the species and strains is given (Table 1). For each nematode species and strain tested, 34-85 individual larvae were utilized. Untreated controls without nematodes were maintained concurrently in similar containers. After introduction of the nematodes, the containers were held at ca. 22° C. for 7-10 days, at which time the root pieces were removed from the sand and dissected to determine the number of GRB larvae infected with nematodes. A binocular microscope was used for root and larval dissection and nematode detection.

TABLE 14

| Nematode | | Year of | | |
|---|---|---|---|---|
| Species | Strain | Isolation | Original Locality | Source |
| H. bacteriophora | GPS2 | 1997 | Jeromesville, OH, USA | Soil |
| H. bacteriophora | GPS11 | 1998 | Atwood, OH, USA | Cyclocephala borealis |
| H. bacteriophora | NC1 | 1975 | Clayton, NC, USA | Helicoverpa zea |
| H. bacteriophora | HP88 | 1982 | Logan, UT, USA | Phylophaga sp. |
| H. bacteriophora | Lewiston | 1993 | Lewiston, NC, USA | Soil |
| H. bacteriophora | Oswego | 1992 | Oswego, NY, USA | Soil |
| H. bacteriophora | OH25 | 1993 | Hermiston, OR, USA | Soil |
| H. bacteriophora | Riwaka | 1990 | Riwaka, New Zealand | Soil |
| H. argentinensis | Argentina | 1993 | Rafaela, Argentina | Graphognathus sp. |
| H. indica | LN2 | 1992 | Coimbatore, India | Soil^{a} |
| H. mamrelata | Oregon | 1996 | Seaside, OR, USA | Soil |
| H. megidis | UK | 1988 | England | Soil |
| H. zealandica | X1 | 1990 | Auckland, New | Heteronychus arator |
| | | | Zealand | |
| S. carpocapsae | All | 1975 | Georgia, USA | Vitacea polistiformis |
| S. bicornutum | Yugoslavia | 1995 | Strazilovo, Yugoslavia | Soil |
| S. glaseri | NC1 | 1977 | North Carolina, USA | Strigoderma |
| | | | | arboricola |
| S. riobrave | RGV | 1990 | Weslaco, TX, USA | Helicoverpa zea |

^{a}Soil baited with Scirpophaga excerptalis larvae.

Statistical Analysis

Nematode strains were ranked by infection frequency and x² goodness of fit was used to compare differences of infection frequency between treatments (Statistica, 1995).

Results

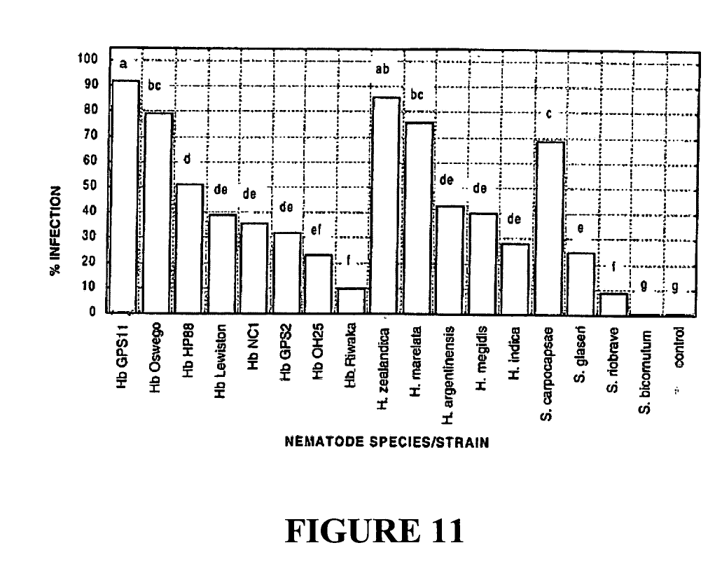

All species and strains of nematodes tested in the laboratory bioassays caused mortality of GRB larvae except S. bicornutum (FIG. 11). Heterorhabditis bacteriophora (GPS11 strain) produced the highest rate of infection at 92%. Statistically, the “GPS11 ” strain was superior to all other strains and species of nematodes tested (x²≧4.43; df=1; P≦0.04) except for H. zealandica, which had an infection rate of 86% (x²=1.14; df=1; P=0.28). In addition, H. bacteriophora Oswego and H. marelata produced about 80% GRB mortality. Of the Steinernema species tested, S. carpocapsae produced an infection rate of 69%, with the remaining species producing zero to low mortality. H. bacteriophora (GPS11 strain) successfully reproduced within the GRB larvae. The number of IJs recovered per infected larva was related to larval size. The general length of first instar larvae ranges from 1.66-2.66 mm. The larvae utilized for nematode reproduction were of second and late instar (Bambara & Neunzig, 1977).

Rearing of newly hatched GRB larvae was an important aspect of this study. Records were kept of the initial number of larvae placed on root pieces and the number of larvae recovered. Nine separate groups of larvae were followed in this manner. Larval survival within these groups ranged from 50 to 72%. A total of 844 neonate larvae were initially placed on root sections and 517 were recovered approximately 6 weeks later. This resulted in a cumulative mean of 61% (±2.94 SE) survival.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise defined, all technical and scientific terms used herein have the same meaning as commonly understood by one of the ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

1. An isolated nematode, wherein the nematode is Heterorhabditis bacteriophora strain GPS11, which was deposited at the ATCC, 10801 University Blvd., Manassas, Va. 20110-2209, on Apr. 22, 2004, under accession number ______, or progeny of said nematode.

2. An insecticidal composition, comprising at least one nematode according to claim 1, and at least one agriculturally acceptable carrier component.

3. The insecticidal composition according to claim 2, wherein the carrier component comprises water.

4. The insecticidal composition according to claim 3, wherein the carrier component further comprises at least one polymer.

5. The insecticidal composition according to claim 4, wherein the polymer is chosen from agaroses, carbopols, carrageenans, dextrins, gums, starches, alginates, acrylamides, and glutens.

6. The insecticidal composition according to claim 3, wherein the carrier component further comprises at least one humectant.

7. The insecticidal composition according to claim 6, wherein the carrier component comprises at least one humectant and at least one polymer.

8. A method of controlling a population of insect pests, comprising applying an effective amount of the nematode according to claim 1, to an affected area.

9. The method according to claim 8, wherein the insect pests to be controlled are chosen from armyworms, cutworms, sod webworms, fleas, mole crickets, crane flies, weevils, white grubs, fungus gnats, and onion maggots.
